(19) United States
(12) Patent Application Publication
(10) Pub. No.: US 2002/0058713 A1
(43) Pub. Date: May 16, 2002

(54) High molecular weight primary aliphatic alcohols obtained from natural products and uses thereof

(76) Inventors: William R. Gamble, Longmont, CO (US); Zhengjie Liu, Superior, CO (US); David T. Bailey, Boulder, CO (US); Pedro P. Perez, Hialeah, FL (US)

Correspondence Address: Steven C. Petersen, Hogan & Hartson, LLP, Suite 1500, 1200 17th Street, Denver, CO, 80202, US

(21) Appl. No.: 09/845,043

(22) Filed: Apr. 27, 2001

Related U.S. Application Data
(63) Continuation-in-part of application No. 09/337,339, filed on Jun. 21, 1999, now Pat. No. 6,225,354.
(60) Provisional application No. 60/236,515, filed on Sep. 29, 2000.

(51) Int. Cl. A61K 31/045; C07C 29/86

(52) U.S. Cl. 514/724; 568/918

(57) ABSTRACT

The present invention relates to a naturally obtained mixture of higher molecular weight primary aliphatic alcohols which contain 20 to 34 carbon atoms. This invention also relates to the process for obtaining the alcohol mixture by extraction and purification with organic solvents from a natural product, such as beeswax with and without saponification of the natural product. The alcohol mixture obtained from beeswax has enhanced purity and contains a mixture of alcohols having 20, 22, 24, 26, 27, 28, 30, 32 and 34 carbon atoms. The alcohol mixture is useful in pharmaceutical compositions, foodstuffs and dietary supplements and is effective for lowering cholesterol in LDL-C levels so that it is effective in treating hypercholesterolemia. Consequently the composition may be used to reduce the risk of coronary heart disease, to inhibit the atherosclerotic process (platelet hyperaggregability, ischemia and thrombosis) and also to act as an anti-inflammatory and anti-thrombotic agent. The composition also possesses neurotrophic properties and is useful for improving male sexual activity.

### CROSS-REFERENCE TO OTHER APPLICATIONS

This patent application is a continuation-in-part application of U.S. patent application Ser. No. 09/337,339, filed Jun. 21, 1999, and entitled “High Molecular Weight Primary Aliphatic Alcohols Obtained From Beeswax and Pharmaceutical use Thereof” and this patent application claims priority from Provisional Application No. 60/236,515 filed on Sep. 29, 2000, and entitled “High Molecular Weight Primary Aliphatic Alcohols Obtained From Beeswax and Uses Thereof.”

### BACKGROUND OF THE INVENTION

1. Field of the Invention

The present invention pertains to a biologically active mixture of primary high molecular weight aliphatic alcohols referred to hereinafter as apisol having enhanced purity that is isolated from a naturally occurring source such as beeswax. More particularly the invention pertains to a highly pure mixture of primary aliphatic alcohols that is obtained from saponified beeswax by liquid extraction wherein the resulting alcohols in the mixture contain 20 to 34 carbon atoms. The C₂₀-C₃₄ alcohols in the mixture advantageously consist of straight chain alcohols having 20, 22, 24, 26, 27, 28, 30, 32 and 34 (i.e., eicosanol, docosanol, tetracosanol, hexacosanol, heptacosanol, octacosanol, triacontanol, dotriacontanol and tetratriacontanol). The invention also pertains to the method of extracting the aforementioned mixture from selected beeswax by a solid-liquid extraction procedure with or without saponification. The invention also pertains to the use of the biological compositions, foodstuffs and dietary supplements for administering the composition.

2. Description of the State of Art

All kinds of waxes, and more especially beeswax, have always been a matter of interest. This has been the case not only because of their industrial application, but also because of their chemical composition. The amount of beeswax in honey ranges between 0.9% to 1.13%, depending on the methods used to separate the wax from the honey. This wax is made up of esters, hydrocarbons, free fatty acids and free alcohols.

The natural mixture of straight chain aliphatic alcohols obtained from beeswax has been studied by several authors to learn about its composition and main features. The obtaining of different mixtures of alcohols from all kinds of waxes has been reported. (J. A. Lamberton, et al., Australian Journal of Chemistry, 13:261-268 (1959) and A. Horn and J. S. Martic, Journal of Science Food and Agriculture, 10:571 (1957) and (Kreger, 1948; Wimbero, 1904; Mitsui and Col, 1942)). These studies suggest a method for obtaining fatty alcohols based on the homogeneous saponification with alcoholic potassium hydroxide, followed by the esterification of the saponified material, and separation of the alcohol.

Another method also reported is extraction of the natural alcohol mixture through a high efficiency vacuum. The high vacuum wax distillation for the chemical isolation of mixed derivatives and the extraction of the remaining wax is done using petroleum ether. The solvent evaporates and the remaining solids are acetylated for further purification through alumina chromatography. Finally, through alkaline hydrolysis, alcohols are obtained and then recrystallized in ethanol, showing a fusion point ranging from 62 to 82 degrees Celsius.

Blood-lipid lowering effects of a natural mixture of straight chain aliphatic alcohols have been demonstrated by several authors (F. Liu, Active Constituents Lowering Blood-Lipid in Beeswax (1996); Chung Kor, Chung Yao Tsa Chih, 21(9) 553-4, 576); (H. Sho, et al, Effects of Okinawa Sugar Cane Wax and Fatty Alcohols on Serum and Liver Lipids in the Rats, J. Nutri Vitaminol, 30(6):553-559 (1984)); (S. Kato, K. Hamatani, et al., Octacosanol Effects Lipid Metabolism in Rat Fed on a High Fat Diet, Br J Nutr, 73(3):433-441 (1995)); (Kabiry et al., Tissue Distribution of Octacosanol in Liver and Muscle of Rats After Serial Administration, Ann Nutr Metab, 39(5):279-284 (1995)). Many investigational studies based on clinical studies with the use of the natural mixture of straight chain aliphatic alcohols have been published.

These studies have demonstrated the characteristics associated with ergogenic effects in humans and animals as well as benefits in the cardiovascular, cerebral and muscular systems. (V. Nataraj an, H. H. Schmid, 1-Docosanol and Other Long Chain Primary Alcohols in Developing Rat Brain, Lipids, 12(1):128-130 (1996)) (M. Azzouz, J. Borg, Enhancement of Mouse Sciatic Nerve Regeneration by the Long Chain Fatty Alcohol, n-Hexacosanol, Exp Neurol, 138(2):189-197 (1996). U.S. Pat. No. 5,447,959, discloses that the long chain fatty alcohols are useful in treating or preventing neuro-degenerative illnesses, conditions linked to skin aging, the phenomena of thrombosis and atherosclerosis, and immune deficiencies. (J. Borg, The Neurotrophic Factor, n-Hexacosanol, Reduces the Neuronal Damage Induced by the Neurotoxin, Kainic Acid, J Neurosci Res, 29(1):62-67 (1991)) (J. Borg, P. J. Kesslak, C. W. Cotman, Peripheral Administration of a Long Chain Fatty Alcohol Promotes Septal Cholinergic Neurons Survival after Fimbria Fomix Transection; 518(1-2):295-298 (1990)) (Y. Kabir, S. Kimura, Distribution of radioactive octacosanol in response to exercise in rats; 38(4):373-377 (1994)) (R. P. Warren, R. A. Burger, R. W. Sidwell, L. L. Clark, Effect of Triacontanol on Numbers and Functions of Cells Involved in Inflammatory Responses, 200(3):349-352 (1992)) (P. W. Westerman, J. M. Pope, N. Phonphok, J. W. Dan, D. W. Dubro, Biochim Biophys Acta (Netherlands), 939:64-78(1988)). Other studies have reported that these alcohols also stimulate growth in plants. Studies have been conducted regarding the partitioning of long-chain alcohols into lipid bilayers. In U.S. Pat. No. 3,031,376, Ezra Levin reported that tetracosanol, hexacosanol, octacosanol and triacontanol and their esters improved physical performance of athletes and disclosed compositions comprising such alcohols and esters in vegetable oil bases for oral ingestion. Various constituents of beeswax and products derived from beeswax have also been used in cosmetic and therapeutic applications, as disclosed by Karen M. Slimak in U.S. Pat. No. 4,793,991 which describes a hypoallergenic cosmetic comprising single plant source beeswax. Gans et al. have described the use of the non-polar saturated straight chain C₂₁ to C₃₃ hydrocarbon fraction of beeswax in the treatment of inflammatory skin disorders in U.S. Pat. No. 4,623,667.

A procedure for obtaining a natural mixture of straight chain higher aliphatic primary alcohols from animal and vegetable wax (a natural source wax) is also known in the prior art. This prior art procedure is based on the extraction of alcohol mixtures with fluid extractant in the sub and supercritical states between 20 and 100 degrees Celsius. Selective extraction can be carried out with this procedure but when this is applied to beeswax it is only possible to obtain between 10% to 15% of C₂₀ to C₃₄ alcohol mixture.

Other projects (S. Inaa, K. Furukama, T. Masui, K. Honda, J. Ogasawara, and G. Tsubikamoto; Process for Recovering Primary Normal Aliphatic Higher Alcohols JP 60-119514 (1996)), proposed a very similar extraction method applied to waxes that is based on fluids (CO₂ with ethylene) in sub and supercritical states.

There are different commercial dietary supplements, foods and drugs to aid in the lowering of total blood cholesterol (lowering lipid, LDL and cholesterol levels) which are considered as effective, safe and well tolerated but most of them produce different adverse side effects. Since lipid-lowering therapy must be chronically administered, safety and tolerableness are very important for their definitive acceptance. Although many products from different sources exist in the market such as β-sitosterol, garlic, bile acid binders, fibric acid derivatives, HMG-Co A reductase inhibitors and nicotinic acid, etc., the methods of use and the quantities necessary of these products are not sufficiently effective for the reduction of cholesterol to the desired levels. In addition, the drugs that are used for the lowering of cholesterol have various adverse side effects.

It has been described that treatment with some lipid-lowering drugs reduces the tendency for platelet hyperaggregation frequently seen in hyperlipidemic patients and experimental data has shown anti-aggregatory effects mediated by these compounds. Nevertheless, only some cholesterol-lowering drugs show this property.

Atherosclerosis is a variable combination of changes of the intima of the arteries consisting of the focal accumulation of lipids, complex carbohydrates, blood and blood products, fibrous tissue and calcium deposits, frequently also associated with medial changes. Thus, atherosclerosis is known as a multifactorial process and includes hyperlipidemia as a risk factor.

Among the factors contributing to atherosclerosis development, platelet aggregation has a very important place. Platelet releasing granule contents activate arachidonic acid, which metabolizes into cyclic endoperoxides. These are mainly transformed into thromboxane A₂ (TXA₂), a strong vasoconstrictor and platelet aggregatory agent. Platelet aggregation can be elicited by numerous compounds, such as collagen, ADP and epinephrine. Thus, different experimental “in vivo”, “ex vivo”, or “in vitro” models testing effectiveness of putative antiplatelet drugs commonly test their effect on platelet aggregation induced by these agents. These tests are also used for testing platelet aggregation in healthy volunteers and in patients with diseases which induce hyperaggregability such as hypercholesterolemia and diabetes.

Collagen-induced platelet aggregation is one of the most frequently used tests. Thus, for example, collagen injected endovenously leads to reversible intravascular platelet aggregation “in vivo” and aggregates of platelet enter the vascular microcirculation, subsequently decreasing the count of circulating platelet and simultaneously increasing the plasma Malondialdehyde (MDA) concentration. Moreover, in some species this injection of collagen induces mortality produced by thrombosis. In these models, antiplatelet drugs generally prevent the decrease in circulating platelet content and increase of MDA concentration, as well as collagen induced mortality.

Some drugs showing platelet anti-aggregatory effects are useful for treatment of thrombotic diseases, myocardial infarction and stroke, but not all show these advantages. On the other hand, there are antithrombotic drugs such as estreptokinase and urokinase that mainly act by lytic processes affecting blood coagulation, but not on the platelet aggregation. Since ischemic cardiovascular diseases, stroke and vascular peripheric obstructive pathologies are the main sequence of atherosclerosis, effects of several drugs on these complications are commonly tested. Thus, theoretically a drug showing cholesterol lowering properties that also can prevent these complications by acting on other events involved in these processes must be advantageous for treating these patients. Likewise, reduction of TXA₂ levels has been associated not only with antiplatelet and antithrombotic effects, but also with antischemic effects. The pharmacological screening of antischemic drugs commonly includes the evaluation of their effects on brain-induced global ischemia. Thus, the protective effect of different drugs on rat cerebral ischemia has been determined by this type of evaluation for certain non-steroidal anti-inflammatory drugs (NSAID) which inhibit reactions catalyzed by cyclooxygenase, as well as for specific inhibitors of thromboxane synthetase and prostacyclin (PGI₂) analogues (M. G. Borzeix and J. Cahn, Effects of new Chemically Metabolically Stable Prostacyclin Analogues on early Consequences of a Transient Cerebral Oligemia in Rats, Prostaglandins, 35(5):653-664 (1998)). Other experimental models, such as global ischemia induced experimentally in Mongolian gerbils are also used frequently.

### SUMMARY OF THE INVENTION

Accordingly, it is an object of this invention to isolate a mixture of higher primary aliphatic alcohols from beeswax.

It is another object of this invention to provide an improved extraction method for obtaining a highly pure mixture of higher primary aliphatic alcohols from beeswax.

It is another object of this invention to improve the purity level of a mixture of higher primary aliphatic alcohols which contains 1-eicosanol, 1-docosanol, 1-tetracosanol, 1-hexacosanol, 1-heptacosanol, 1-octacosonal, 1-triacontanol, 1-dotriacontanol, and 1-tetratriacontanol therein.

It is another object of this invention to improve the percent recovery of the aforementioned mixture of C₂₀-C₃₄ primary aliphatic alcohols obtained by solid-liquid extraction from beeswax.

It is another object of this invention to provide a composition which contains a highly pure mixture of C₂₀-C₃₄ primary aliphatic alcohols as an active ingredient by itself or in combination with other active ingredients.

It is another object of this invention to provide an improved mixture of higher (C₂₀-C₃₄) primary aliphatic alcohols which can be used to lower cholesterol levels.

Additional objects, advantages and novel features of the invention shall be set forth in part in the description and examples that follow, and in part will become apparent to those skilled in the art upon examination of the following specification or may be learned by practice of the invention. The objects and advantages of the invention may be realized and attained by means of the instrumentalities, combinations, compositions, and methods particularly pointed out in the appended claims.

To achieve the foregoing and other objects and in accordance with the purposes of the present invention, as embodied and broadly described therein the composition of the present invention is obtained by initially subjecting beeswax to a homogenous phase saponification step after which the saponified beeswax is recovered, dried and ground to a particle mesh size of 120-500 microns.

Next, the particles of saponified beeswax are placed into a conventional solid-liquid extractor and an organic solvent is then introduced and contacted with the beeswax particles. The resulting extract is then maintained within the temperature range of 2° C-1° C. causing the alcohols to solidify and form a suspension. The suspension is then filtered and the solids are recovered and air dried. The dried solid obtained from the drying step is then sent to a purifier where it is contacted with and dissolved in another solvent. This solution is then chilled, and the solids collected dried by vacuum. The dried solids obtained from the purification step are then contacted with hot organic solvent, which dissolves it. This solution is then hot filtered, chilled to resolidify the solids, collected, dried and powdered.

The final product contains a mixture of higher primary aliphatic alcohols from 20 to 34 carbon atoms comprising 1-eicosanol, 1-docosanol, 1-tetracosanol, 1-hexacosanol, 1-heptacosanol, 1-octacosanol, 1-triacontanol, 1-dotriacontanol, and 1-tetratriacontanol having the following quantitative composition:

| | | |
|---|---|---|
| 1-eicosanol | C-20 | 0.0-5.0% |
| 1-docosanol | C-22 | 0.0-5.0% |
| 1-tetracosanol | C-24 | 15.0-30.0% |
| 1-hexacosanol | C-26 | 15.0-30.0% |
| 1-heptacosanol | C-27 | 0.0-5.0% |
| 1-octacosanol | C-28 | 12.0-22.0% |
| 1-tricontanol | C-30 | 20.0-35.0% |
| 1-dotriacontanol | C-32 | 5.0-15.0% |
| 1-tetratriacontanol | C-34 | 0.0-5.0% |

and may be used as is or reformulated for administration to humans and animals to reduce and prevent hyper-cholesterolemic diseases, cholesterol, coronary heart disease (heart attacks and strokes), inflammation or immunoregulatory diseases, cardiovascular diseases, and neurodegenerative disorders. The daily dosage is established between 1 to 100 mg per day (preferably 3 to 20 mg) and is intended for ingestion in any type of form of foodstuff, capsule, tablet or liquid form.

### BRIEF DESCRIPTION OF THE DRAWINGS

The accompanying drawings, which are incorporated in and form a part of the specification, illustrate the preferred embodiments of the present invention, and together with the description serve to explain the principles of the invention.

In the Drawings

FIG. 1 is a flow diagram depicting the saponification step of the process according to the present invention.

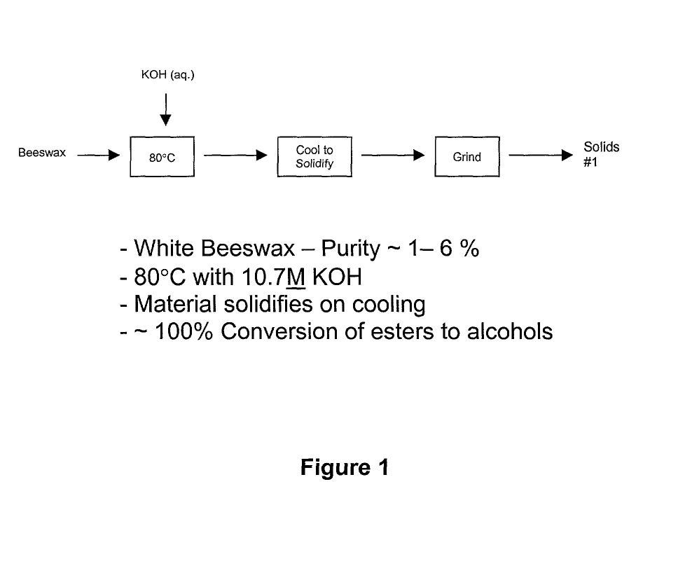

FIG. 2 is a flow diagram depicting the extraction step of the process according to the present invention.

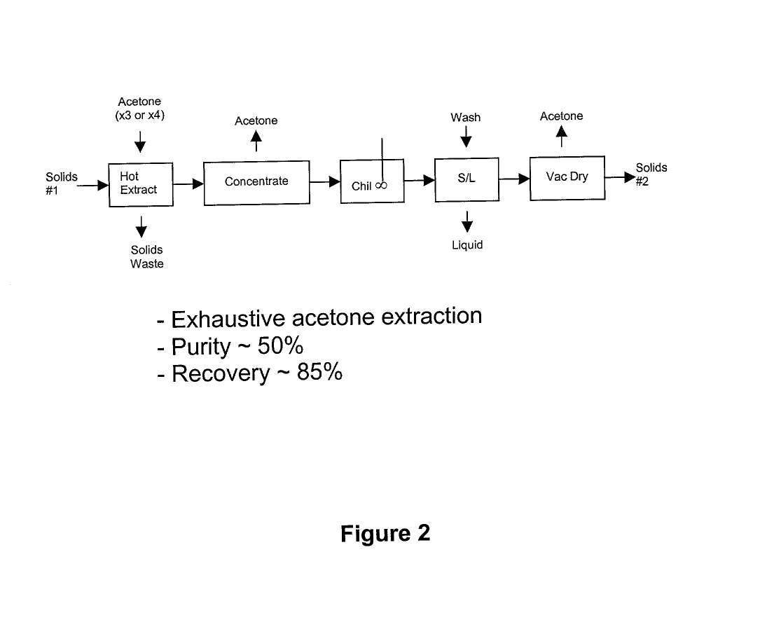

FIG. 3 is a flow diagram depicting the first purification step of the process according to the present invention.

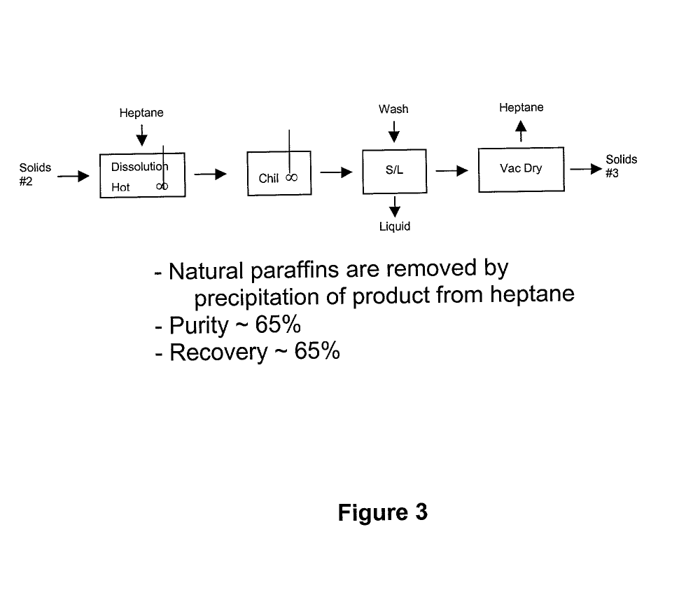

FIG. 4 is a flow diagram depicting the second purification step of the process according to the present invention.

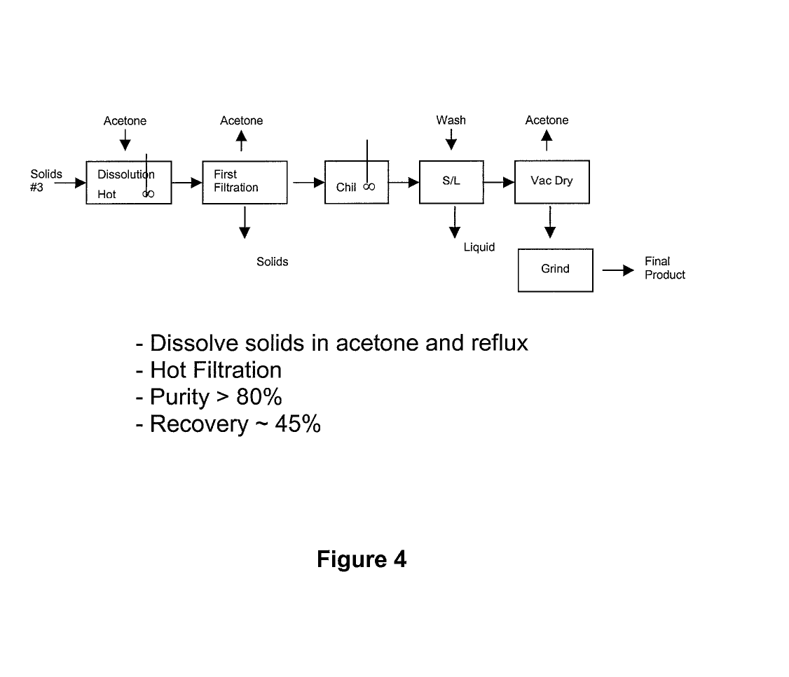

FIG. 5 is a flow diagram depicting the theoretical yields as a result of the saponification step of the process according to the present invention.

FIG. 6 shows the range of total apisol content for all below processed materials.

### DETAILED DESCRIPTION OF INVENTION AND PREFERRED EMBODIMENTS

The apisol composition of the present invention is obtained by saponifying, extracting and purifying from starting materials such as but not limited to, waxes, such as but not limited to, beeswax; carnauba wax, and candellia wax; bee pollen; oils, such as but not limited to, peanut oil, sesame oil, Norwegian cod liver oil, and rice bran oil; rosemary needles; and powders, such as but not limited to rice bran and carnauba wax powder, the natural mixture of straight chain primary aliphatic alcohols (higher aliphatic primary alcohols). For purposes of the remainder of this discussion beeswax is used to typify the starting material, however, it should be understood that the process of the disclosed invention and the compositions may be utilized with the above referenced starting materials. The beeswax is initially subjected to a homogenous phase saponification step after which the saponified beeswax is ground to a particle mesh size of 120-500 microns. Next, the particles of saponified beeswax are placed into a conventional solid-liquid extractor and an organic solvent is then introduced and contacted with the beeswax particles.

The resulting extract is then maintained within the temperature range of 2° C.-10° C. causing the alcohols to solidify and form a suspension. The suspension is then filtered and the solids are recovered and air dried. The dried solid obtained from the drying step is then sent to a purifier where it is contacted with and dissolved in another solvent. This solution is then recrystallized and dried by vacuum. The dried solids obtained from the purification step are then contacted with hot organic solvent, which dissolves it. This solution is then hot filtered, chilled to recrystallize solids, collected, dried and powdered.

The final product contains a mixture of higher primary aliphatic alcohols from 20 to 34 carbon atoms comprising 1-eicosanol, 1-docosanol, 1-tetracosanol, 1-hexacosanol, 1-heptacosanol, 1-octacosanol, 1-triacontanol, 1-dotriacontanol, and 1-tetratriacontanol having the following quantitative composition:

| | | |
|---|---|---|
| 1-eicosanol | C-20 | 0.0-5.0% |
| 1-docosanol | C-22 | 0.0-5.0% |
| 1-tetracosanol | C-24 | 15.0-30.0% |
| 1-hexacosanol | C-26 | 15.0-30.0% |
| 1-heptacosanol | C-27 | 0.0-5.0% |
| 1-octacosanol | C-28 | 12.0-22.0% |
| 1-tricontanol | C-30 | 20.0-35.0% |
| 1-dotriacontanol | C-32 | 5.0-15.0% |
| 1-tetratriacontanol | C-34 | 0.0-5.0% |

This product has been developed for use in lowering LDL-C and total cholesterol and improving LDL/HDL ratios.

It has been discovered that the use of beeswax as the source of the alcohol mixture offers numerous benefits which cannot be obtained when sugar cane wax is used as the source. For example, many factors are associated with purity levels of sugar cane wax which are difficult to control and thus it is difficult to obtain a reliable source of sugar cane wax which meets the requirements for consistently obtaining a uniform pure product.

In particular the purity level of sugar cane wax is influenced by the sugar cane variety, age of the plant, soil and climate conditions where the sugar cane is grown and the level and type of fertilizer used to grow the sugar cane. In addition the type of operational procedure used to extract the wax from the husk can influence the level of purity. None of these factors are significant when beeswax is used as the source for the alcohol mixture. Furthermore, minor variations in beeswax characteristics can be corrected by blending of selected waxes which meet certain criteria with respect to parameters as further described herein.

Furthermore, preferred levels of certain operational parameters in the saponification, extraction and purification process have been discovered which lead to further enhancement of the purity level of the isolated alcohols and enhanced percent recovery of the alcohols from the beeswax. These operational parameters include one or more of the following, particle size of the solid (i.e., the particle size of the beeswax), alkali concentration, relationship between solid and liquid (i.e., solid:liquid ratio), temperature range, fluid regimen, crystallization regimen, hot filtration regimen, centrifuge regimen and contact time. The preferred levels of these parameters were established under experimental design in the laboratory as well as at the pilot plant and industrial level.

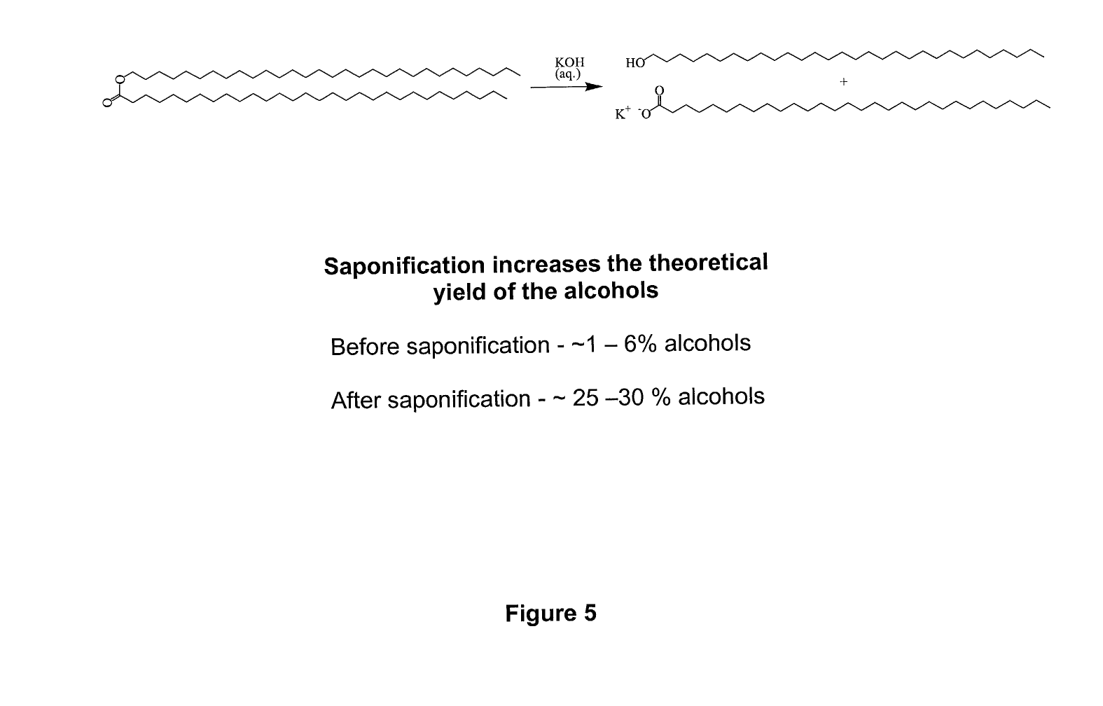

The procedure for a homogeneous phase saponification process of beeswax, in the present invention consists of melting beeswax at a temperature of 80° C.-100° C., to which an aqueous solution of potassium hydroxide (10.7 M) is added with continuous stirring at 40-100 rpm, to the previously melted beeswax. The saponification process is continued for 3 hours with continuous stirring at 80 rpm. It has been determined that each pound of wax requires between 38 grams and 47 grams of hydrogen peroxide for complete saponification. The homogeneous phase saponification process of beeswax in the present invention increases the theoretical yield of the alcohols as shown in FIG. 5.

In a preferred embodiment the saponified beeswax is ground to achieve a particle size of 125-500 microns in diameter, preferably 250 microns in diameter. The particles of wax are placed into a conventional solid-liquid extractor. An organic solvent extractant is also introduced into the extractor for contact with the particles of beeswax contained therein. Preferably acetone is used as the extractant. However, other examples of suitable solvents, such as but not limited to pentanone, toluene, benzene, ethanol, heptane, propanol, isopropanol, ethylacetate, methanol, hexane, n-butanol, phenol, ether, trichloroethane, methyl ethyl ketone, 1,2-dichloroethane, dichloromethane, chloroform and mixtures thereof may also be used. The ratio of beeswax particles to liquid extractant is preferably from 1:4 to 1:8, preferably 1:6. The extraction is conducted for 3 to 7 hours, preferably 4 hours within a temperature range of 50° C.-60° C., preferably 56° C. The saponified beeswax particles are preferably agitated during the extraction procedure, for example by use of a rotating agitator to agitate the particles while in contact with the solvent. Advantageously the agitator is rotated at 40-100 rpm, preferably 80 rpm. During the extraction procedure the alcohols become solubilized in the extractant thus leaving a waxy residue. Upon completion of the extraction, the extractant containing the alcohols dissolved therein is removed from the waxy residue.

Next the extract is introduced into a chamber for solidifying the alcohols. The alcohols are advantageously solidified by reducing the temperature of the extract in the chiller to form solids in the extractant (e.g., an alcohol-extract suspension or mixture). Preferably the temperature in the chiller should be uniform. An agitator may be provided within the crystallizer to assure a uniform temperature therein. The agitator is rotated at 40-80 rpm, preferably 60 rpm. The temperature during solidification is maintained within the range of 2° C.-1 0° C., preferably 6° C.

The suspension or mixture obtained from the chiller is then vacuum filtered or centrifuged to recover the solids. The solids are routinely washed. Centrifugation takes place by initially centrifuging the mixture or suspension at 80 rpm and gradually increasing the revolutions per minute to 2,000 rpm over a 2 hour period of time. During centrifugation the particles may be washed with a spray of clean extractant for about 1-2 minutes to remove contaminating material which may be contained in the mother liquid.

The clean solid mixture obtained from the centifugation step is then recovered and dried. Vacuum drying may be used. A pressure of 400 millibars at a temperature of 50° C. may be used during the vacuum drying step.

The dried solid obtained from the vacuum drying step is then sent to a purifier where it is contacted with another solvent which is preferably hot heptane. The alcohol solids are dissolved in the heptane to form a solution. The solution is then introduced into a chiller for solidification. The solidification is performed under the same conditions as the initial solidification from the acetone solution. Thus, the same type of chiller may be used in the resolidification step as was used in the initial solidification from the acetone. The temperature of the heptane solution is kept uniform by agitating with an agitator at 40-80 rpm preferably 60 rpm. The temperature during resolidification is maintained at 2° C.-10° C., preferably 6° C. The resolidification step forms a mixture or suspension of solids in the heptane solvent.

The suspension or mixture of solids in the heptane is then introduced into a filtration device where it is filtered in the same manner that the solids mixture was filtered from the acetone solvent. During this second filtration step, the solids are washed for 1-2 minutes with clean solvent (heptane).

The washed particles obtained from the second filtration step are then recovered and dried. Vacuum drying may be used. A pressure of 400 millibars at a temperature of 50° C. may be used during the vacuum drying step.

The dried solid obtained from the heptane purification step is then sent to a purifier where it is contacted with another solvent which is preferably hot acetone. The crystals are dissolved in the acetone. The hot acetone solution is then passed through a hot filtration system. The acetone solution is then introduced into a chiller for resolidification. The solidification is performed under similar conditions as the initial solidification of the alcohols from the acetone solution. Thus the same type of chiller may be used in the solidification step. The temperature of the acetone solution is kept uniform preferably by agitating with an agitator at 40-80 rpm, preferably 60 rpm. The temperature during resolidification is maintained at 2° C.-10° C., preferably 6° C. The resolidification step forms a mixture or suspension of alcohol crystals in the acetone solvent.

The suspension or mixture of solids in the acetone is then introduced into a filter, where it is filtered in the same manner that the solid suspension or mixture was filtered from the first acetone solvent. During this third filtration step, the crystals are washed for 1-2 minutes with clean solvent (acetone). The washed particles obtained from the third Siltation step are then recovered and dried. Vacuum drying may be used. A pressure of 400 millibars at a temperature of 35° C. may be used during the vacuum drying step.

After the particles are dried, they are then ready to be formulated into a conventional pharmaceutical formulation such as tablets, capsules, etc., for administration.

The yield (i.e., percent recovery by weight of alcohols with respect to the weight of alcohols in saponified beeswax) attained ranges about 50% with purity ranges from 80 to 99%. The natural mixture obtained contains alcohols ranging from 20 to 34 carbon atoms, with melting point between 61 and 65 degrees Celsius. The natural mixture of straight chain aliphatic alcohols obtained by this process may be analyzed through gas chromatography in Fused Silica Capillary Column.

The procedure of this invention for obtaining the natural mixture of higher primary molecular weight aliphatic alcohols from beeswax has some advantages compared to other prior art procedures.

Advantages of this invention are related to the practical yields (10-15% by weight) compared with the previously reported results with yields lower than 5%. Another advantage of the procedure relates to the purity that can be obtained (80-99%) which is significantly higher than the purity in the prior art methods. Thus, the method of the present invention is simple and appropriate for large scale production.

The composition of the invention has new surprising pharmaceutical properties including antiplatelet, anti-inflammatory, anti-thrombotic and antischemic properties. In addition the composition is useful in the prevention of foam cell development, and the treatment of hypercholesterolemia; it also provides a protective effect on the vascular endothelium and can be used for the prevention of early atherosclerotic lesions (thrombus formation). The composition also demonstrates neurotrophic properties.

The pharmaceutical composition, foodstuffs, and dietary supplements formulated with the natural mixture of higher aliphatic primary alcohols of this invention may be administered to humans and animals. The daily dosage of this natural mixture obtained from beeswax to be used for the reduction and prevention of hyper-cholesterolemic diseases, cholesterol, coronary heart disease (heart attacks and stroke), inflammation or immunoregulatory diseases, cardiovascular disease and neurodegenerative disorders is established between 1 to 100 mg per day (preferably 3 to 20 mg) and is intended for ingestion in any type or form of foodstuff, capsule, tablet or liquid form.

The below-described examples further describe the invention and preferred embodiments thereof.

### EXAMPLES

Saponification:

Seven kg of wax was heated using a water bath at 80-85° C. for 2.5 hours until completely melted. Once the wax was completely melted, 1.22 L of 10.7 M KOH in water was added dropwise over 30 minutes while continuing the stirring and heating. The mixture was held at temperature for 3 hours with stirring. After 3 hours the saponified wax was poured into trays and dried in a 60-65° C vacuum oven. The 7.54 kg of dried saponified wax was then ground using a Cuisinart blender.

Extraction:

The saponified solids were placed in a 72 L round bottom flask along with 45 L of acetone and the acetone was brought to relux and held there for 3 hours. The heat was then removed and the solids allowed to settle. The extract was decanted through cheese cloth into 5-gallon buckets. Forty-five liters of fresh acetone were added to the solids in the round bottom flask and the extraction and decantation repeated to generate a total of 3 extracts. The extracts were chilled at 4-8° C. for 40-45 hrs and the solids recovered by filtration using Buchner funnels and Whatman #4 filter paper. The 2.78 kg of collected solids were air dried for 48 hr.

Heptane Purification:

The acetone extract solids were dissolved in 60 L of heptane by holding the solution at 55-65° C. for 2 hr with stirring. The hot solution was poured into five-gallon buckets which were sealed and chilled for 27 hr. The solutions were then filtered using Buchner funnels with 50 μm porosity monofilament nylon cloth. The solids in the Buchner funnels were rinsed with an additional 1.5 L of fresh heptane and the solids dried in a vacuum oven at 45° C. and 15 in. Hg to provide 1.9 kg of heptane solids.

Acetone Purification:

The 1.9 kg of heptane solids were extracted with 36 L of acetone at reflux over 2 hr. This extract was hot filtered, decanted, and put into sealed buckets. An equal volume of fresh acetone was added and the extraction repeated for an additional 2 hr. The extracts were chilled at ˜5° C. for 42 hr and then the solid precipitate collected on Buchner funnels using Whatman #4 filter paper. The solids in the Buchner funnels were rinsed with an additional 1.5 L of fresh acetone and then dried in a 35° C. vacuum oven for 17 hr to provide 0.942 kg of product.

The final product described above was used in an open label, single center study to evaluate the effectiveness and tolerability of apisol at lowering LDL-C, total cholesterol, and improving LDL/HDL ratio, the results of which are summarized below, involving 14 subjects who took 10 mgs of apisol per day for 6 weeks. There were no controls on the subjects diets. All data is reported in mg/dL.

TABLE 1
CHOLESTEROL

| SUBJECT | T1 | T2 | % Chg | T3 | % Chg |
|---|---|---|---|---|---|
| 1 | 218 | 243 | 11% | 229 | 5% |
| 2 | 200 | 216 | 8% | 212 | 6% |
| 3 | 170 | 150 | −12% | 157 | −8% |
| 4 | 207 | 155 | −25% | 171 | −17% |
| 5 | 256 | 217 | −15% | | |
| 6 | 206 | 196 | −5% | | |
| 7 | 314 | 253 | −19% | 268 | −15% |
| 8 | 176 | 167 | −5% | 171 | 3% |
| 9 | 161 | 164 | 2% | | |
| 10 | 156 | 167 | 7% | 173 | 11% |
| 11 | 240 | | | 220 | −8% |
| 12 | 212 | 185 | −13% | 184 | −13% |
| 13 | 260 | 212 | −18% | 224 | −14% |
| 14 | 231 | 208 | −10% | 241 | 4% |
| Average | 215 | 195 | −9% | 205 | −5% |
| | p* | 0.06 | | 0.52 | |

T1 = Baseline (before taking apisol)
T2 = 3 weeks of 10 mgs apisol per day
T3 = 6 weeks of 10 mgs apisol per day
*Comparison with baseline by Wilcoxon statistical analysis
**Cardiovascular Disease Risk (CDR) = Cholesterol/HDL

TABLE 2
TRIGLYCERIDES

| SUBJECT | T1 | T2 | % Chg | T3 | % Chg |
|---|---|---|---|---|---|
| 1 | 162 | 143 | −12% | 211 | 30% |
| 2 | 64 | 100 | 56% | 63 | −2% |
| 3 | 97 | 128 | 32% | 109 | 12% |
| 4 | 428 | 203 | −53% | 300 | −30% |
| 5 | 198 | 108 | −45% | | |
| 6 | 96 | 111 | 16% | | |
| 7 | 270 | 238 | −12% | 204 | −24% |
| 8 | 68 | 74 | 9% | 67 | −1% |
| 9 | 69 | 92 | 33% | | |
| 10 | 82 | 109 | 33% | 108 | 32% |
| 11 | 48 | | | 99 | 106% |
| 12 | 213 | 220 | 3% | 196 | −8% |
| 13 | 125 | 186 | 49% | 185 | 48% |
| 14 | 155 | 119 | −23% | 118 | −24% |
| Average | 148 | 141 | −5% | 151 | 2% |
| | p* | 1.00 | | | 0.97 |

T1 = Baseline (before taking apisol)
T2 = 3 weeks of 10 mgs apisol per day
T3 = 6 weeks of 10 mgs apisol per day
*Comparison with baseline by Wilcoxon statistical analysis
**Cardiovascular Disease Risk (CDR) = Cholesterol/HDL

TABLE 3
HDL

| SUBJECT | T1 | T2 | % Chg | T3 | % Chg |
|---|---|---|---|---|---|
| 1 | 41 | 42 | 2% | 40 | −2% |
| 2 | 48 | 45 | −6% | 60 | 25% |
| 3 | 32 | 31 | −3% | 33 | 3% |
| 4 | 38 | 41 | 8% | 49 | 29% |
| 5 | 59 | 69 | 17% | | |
| 6 | 50 | 51 | 2% | | |
| 7 | 61 | 54 | −11% | 66 | 8% |
| 8 | 49 | 45 | −8% | 54 | 10% |
| 9 | 54 | 49 | −9% | | |
| 10 | 60 | 56 | −7% | 71 | 18% |
| 11 | 63 | | | 62 | −2% |
| 12 | 42 | 35 | −17% | 41 | −2% |
| 13 | 42 | 34 | −19% | 38 | −10% |
| 14 | 61 | 57 | −7% | 63 | 3% |
| Average | 50 | 47 | −6% | 52 | 5% |
| | p* | 0.11 | | 0.1 | |

T1 = Baseline (before taking apisol)
T2 = 3 weeks of 10 mgs apisol per day
T3 = 6 weeks of 10 mgs apisol per day
*Comparison with baseline by Wilcoxon statistical analysis
**Cardiovascular Disease Risk (CDR) = Cholesterol/HDL

TABLE 4
LDL

| SUBJECT | T1 | T2 | % Chg | T3 | % Chg |
|---|---|---|---|---|---|
| 1 | 144.6 | 172.4 | 19% | 147.0 | 2% |
| 2 | 139.2 | 151.0 | 8% | 139.0 | 0% |
| 3 | 118.6 | 93.0 | −22% | 102.0 | −14% |
| 4 | N/A | 73.4 | | 62.0 | |
| 5 | 157.4 | 126.0 | −20% | | |
| 6 | 136.8 | 122.8 | −10% | | |
| 7 | 199.0 | 151.4 | −24% | 161.0 | 19% |
| 8 | 113.4 | 107.2 | −5% | 104.0 | −8% |
| 9 | 93.2 | 96.6 | 4% | | |
| 10 | 79.6 | 89.2 | 12% | 80.0 | 1% |
| 11 | 167.4 | | | 138.0 | −18% |
| 12 | 127.4 | 106.0 | −17% | 104.0 | −18% |
| 13 | 183.0 | 140.8 | −23% | 149.0 | −19% |
| 14 | 139.0 | 127.2 | −8% | 154.0 | 11% |
| Average | 138.0 | 120 | −13% | 122.0 | −12% |
| | p* | 0.11 | | 0.08 | |

T1 = Baseline (before taking apisol)
T2 = 3 weeks of 10 mgs apisol per day
T3 = 6 weeks of 10 mgs apisol per day
*Comparison with baseline by Wilcoxon statistical analysis
**Cardiovascular Disease Risk (CDR) = Cholesterol/HDL

TABLE 5
Cardiovascular Disease Risk (CDR)

| SUBJECT | T1 | T2 | % Chg | T3 | % Chg |
|---|---|---|---|---|---|
| 1 | 5.32 | 5.79 | 9% | 5.73 | 8% |
| 2 | 4.17 | 4.80 | 15% | 3.53 | −15% |
| 3 | 5.31 | 4.84 | −9% | 4.76 | −10% |
| 4 | 5.45 | 3.78 | −31% | 3.49 | −36% |
| 5 | 4.34 | 3.14 | −28% | | |
| 6 | 4.12 | 3.84 | −7% | | |
| 7 | 5.15 | 4.69 | −9% | 4.06 | −21% |
| 8 | 3.59 | 3.71 | 3% | 3.17 | −12% |
| 9 | 2.98 | 3.35 | 12% | | |
| 10 | 2.60 | 2.98 | 15% | 2.44 | −6% |
| 11 | 3.81 | | | 3.55 | −7% |
| 12 | 5.05 | 5.29 | 5% | 4.49 | −11% |
| 13 | 5.92 | 6.24 | 5% | 5.89 | −1% |
| 14 | 3.79 | 3.65 | −4% | 3.83 | 1% |
| Average | 4.40 | 4.32 | −2% | 4.09 | −7% |
| | p* | 0.11 | 0.26 | 0.08 | 0.02 |

T1 = Baseline (before taking apisol)
T2 = 3 weeks of 10 mgs apisol per day
T3 = 6 weeks of 10 mgs apisol per day
*Comparison with baseline by Wilcoxon statistical analysis
**Cardiovascular Disease Risk (CDR) = Cholesterol/HDL

As demonstrated by the data in the above tables there was a 12% reduction in LDL that is statistically significant at 91.6% confidence level. This is 1% less then was seen at the 3 weeks, 13% reduction and the most likely explanation is the lack of a controlled diet. It is important to note that the 2^{nd} 3 weeks of testing, weeks 4-6, coincided with the July 4^{th} holiday. This is typically a vacation period for most individuals and eating habits tend not to follow normal behavior. Overeating, lack of exercise, and higher fat foods tend to be the norm for July 4^{th} and vacation.

The 5% reduction in total cholesterol is not statistically significant. While the 3 week data had shown a 9% reduction and was statically significant at 94% confidence level, the 6 week data dose does not support this trend. Again, the most likely explanation is that of diet.

It is important to note that with an uncontrolled diet it is not uncommon to see an increase in cholesterol and LDL-C over the course of the study. This has been demonstrated in the placebo group in other apisol studies. See, Canetti, M., et. al., A two-year study on the efficacy and tolerability of apisol in patients with type ii hyperlipoproteinaemia. Int. J Clin. Pharm. Res., XV (4),159-165 (1995).

A 5% increase in HDL-C levels, 89% confidence level, was observed and this was not unexpected. Prior studies in humans have shown an increase in HDL-C levels.

The CDR was reduced by 7%, 98% confidence level. This is due to the decrease cholesterol and increase HDL-C levels and was expected.

There was no improvement in triglycerides and this is not unexpected. No prior studies on apisol have indicated a reduction in triglycerides.

The data looks promising. Previous studies of apisol, sugar cane material, by Pons P., et. al, Effects of successive dose increases of poliocosanol on the lipid profile of patients with type II hypercholesterolaemia and tolerability to treatment. Int. J Clin. Pharm. Res., XIV (1), 27-33 (1994), demonstrated an 8% reduction in total cholesterol in 8 weeks with 5 mg/day and a 14.1% reduction in total cholesterol in 16 weeks with 5 mg/day for the first 8 weeks and than 10 mg/day for the remaining 8 weeks. They also reported a 11.3% and 21.9% reduction in LDL-C from the same study. This data is consistent with the results of the current study and one would expect to see further reduction in both LDL-C and total cholesterol if the study duration were continued.

### Alternate Wax Sample Preparation

For wax samples (such as Candellia wax or Carnauba wax) each was accurately weighed (20 g) into an 80-mL beaker and placed into a 95° C. oil bath. Slowly 3.5 mL of 10.7 M KOH was added to the melted wax. The sample was saponified for 3 hours with stirring and air dried for over 18 hours at room temperature. The dried saponified wax was ground into small pieces using a coffee grinder. An 18 g sample was accurately weighed and transferred to a 25×100 mm extraction thimble, and then soxhlet extracted with 126 mL acetone for 5 hours.

### Alternate Oil Sample Preparation

For oil samples (such as Peanut oil, Sesame oil, Norwegian Cod Liver oil and Rice Bran oil), each was accurately weighed (20 g) into an 80-mL beaker and placed into a 95 ° C. oil bath. Slowly 3.5 mL of 10.7 M KOH was added with stirring. The sample was saponified for 3 hours. The oily saponified material was air dried over night and chilled at 0 ° C. for about 5 hours. Twenty grams of the sample were quickly weighed into a 25×100 mm extraction thimble and soxhlet extracted with 200 mL acetone for 5 hours.

### Alternate Powder Sample Preparation

For powder samples (such as Rice Bran or powder from uncapped Bee Pollen capsules), twenty-two grams of each was accurately weighed into a 100-mL beaker and placed into a 95 ° C. oil bath. Slowly 3.9 mL of 10.7 M KOH and 45 mL DI water were added with stirring. The samples were saponified for 3 hours and air dried for about 18 hours. The saponified wax was broken into small pieces using a coffee grinder. Each sample was weighed accurately and 20 g transferred into a 25×100 mm extraction thimble and soxhlet extracted with 250 mL acetone for 5 hours.

### Alternate Needle Sample Preparation

Thirty grams of Rosemary needles were weighed into a 48×123-mm extraction thimble. Heptane 300-mL was added to a 500 mL round bottom flask, the RB flask connected to a Soxhlet extraction system and refluxed for 5 hours. The heptane extract was evaporated by rotary evaporator at 50° C. to dryness. The solids were then heated in a 95° C. oil bath and 15 mL DI water added to make a slurry. Saponification was carried out by slowly adding 0.4mL of 10.7 M KOH and holding at temperature for 3 hours. The saponified solids obtained were freeze-dried for about 18 hours and broken into small pieces using a coffee grinder. The sample was weighed accurately and 2 g transferred into a 25×100 mm extraction thimble, and soxhlet extracted with 100 mL acetone for 5 hours.

### Apisol analysis

All acetone extracts were separately evaporated to dryness without exceeding 50 ° C. The samples were analyzed for total apisol content by GC-FID.

Results

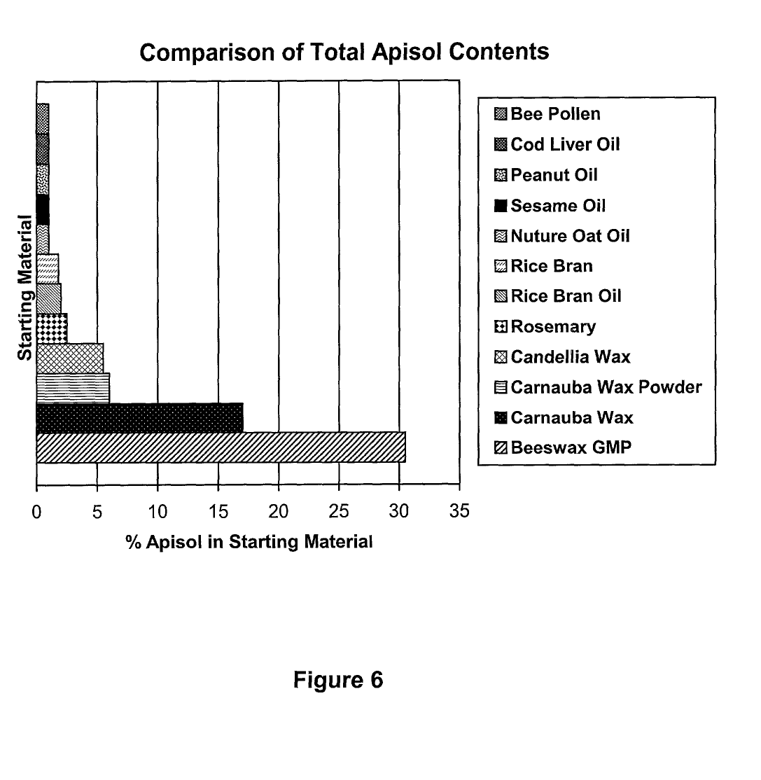

The percentage of apisol in the starting material was calculated based on the total apisol content in each acetone extract. The range of apisol contents for all above processed materials is shown in the FIG. 6. Gas chromatography analysis for individual apisol percentages are listed in Table 6, below. The data for apisol from beeswax is from the current GMP process. The most 10 apisol was found in Beeswax, which was as high as 30%. Carnauba wax contained about 17%, Candellia Wax about 5%, Rosemary about 3% and Rice Bran oil 2% apisol. All other samples assayed showed less than 1% apisol in the starting material.

TABLE 6

| | | | Apisol | | | | | | | | | |
|---|---|---|---|---|---|---|---|---|---|---|---|---|
| | | Apisol | % in | | | | | | | | | |
| Source | | % in Acetone | Starting m | Percentage | | | | | | | | |
| Starting Material | Exp # | Extract | Material | C20 | C22 | C24 | C26 | C27 | C28 | C30 | C32 | C34 |
| Bee Pollen | 2360-176 | 2.5 | 0.12 | 8 | 19 | 13 | 11 | 3 | 0 | 27 | 8 | 0 |
| Rice Bran Powder | 2360-181 | 0.8 | 0.17 | 3 | 2 | 3 | 2 | 2 | 11 | 68 | 8 | 0 |
| Rosemary | 2360-190 | 47.9 | 2.6 | 0 | 0 | 0 | 7 | 2 | 68 | 16 | 8 | 0 |
| Carnauba Wax | 2424-35 | 49.2 | 16.9 | 0 | 0 | 3 | 0 | 0 | 5 | 16 | 77 | 0 |
| Candella Wax | 2424-37 | 8.5 | 5.3 | 0 | 0 | 0 | 5 | 13 | 16 | 42 | 24 | 0 |
| Nuture Oat Oil | 2424-39 | 1.0 | 0.95 | 36 | 0 | 0 | 0 | 0 | 0 | 54 | 0 | 0 |
| Rice Bran Oil | 2424-41 | 2.0 | 1.86 | 5 | 14 | 8 | 5 | 7 | 14 | 27 | 18 | 0 |
| Peanut Oil | 2424-47 | 0.2 | 0.06 | 0 | 0 | 0 | 0 | 0 | 0 | 100 | 0 | 0 |
| Sesame Oil | 2424-49 | 0.3 | 0.15 | 0 | 0 | 0 | 0 | 0 | 0 | 100 | 0 | 0 |
| Carnauba Wax | 2424-54 | 21.1 | 5.6 | 0 | 0 | 0 | 0 | 0 | 50 | 21 | 74 | 0 |
| Cod Liver Oil | 2424-55 | 0.2 | 0.14 | 0 | 0 | 0 | 0 | 0 | 100 | 0 | 0 | 0 |
| Saponified Beeswax | | 51.0 | 30.6 | 0 | 0 | 18 | 17 | 0 | 19 | 31 | 15 | 0 |
| GMP RUN #1-14 | | | | | | | | | | | | |

The foregoing description is considered as illustrative only of the principles of the invention. Further, since numerous modifications and changes will readily occur to those skilled in the art, it is not desired to limit the invention to the exact construction and process shown as described above. Accordingly, all suitable modifications and equivalents may be resorted to falling within the scope of the invention as defined by the claims that follow.

The words “comprise,” “comprising”, “include,” “including,” and “includes” when used in this specification and in the following claims are intended to specify the presence of stated features, integers, components, or steps, but they do not preclude the presence or addition of one or more other features, integers, components, steps, or groups thereof.

The embodiments of the invention in which an exclusive property or privilege is claimed are defined as follows:

1. A method for isolating a mixture of primary aliphatic alcohols from nonalcoholic compounds contained in natural wax wherein said method comprises subjecting saponified beeswax to liquid extraction with a liquid organic extractant in which said alcohols are soluble; recovering said alcohol mixture from said extractant whereby said alcohol mixture is isolated from said nonalcoholic compounds contained in said beeswax and said isolated alcohol mixture includes:

| | | |
|---|---|---|
| 1-eicosanol | C-20 | 0.0-5.0% |
| 1-docosanol | C-22 | 0.0-5.0% |
| 1-tetracosanol | C-24 | 15.0-30.0% |
| 1-hexacosanol | C-26 | 15.0-30.0% |
| 1-heptacosanol | C-27 | 0.0-5.0% |
| 1-octacosanol | C-28 | 12.0-22.0% |
| 1-tricontanol | C-30 | 20.0-35.0% |
| 1-dotriacontanol | C-32 | 5.0-15.0% |
| 1-tetratriacontanol | C-34 | 0.0-5.0% |

2. The method of claim 1 wherein said liquid organic extractant is selected from the group comprising: acetone, toluene, benzene, ethanol, heptane, hexane, pentanone, methanol, phenol, propanol, isopropanol, ethyl acetate, ether, trichloroethane, methyl ethyl ketone, butanol, 1,2-dichioroethane, dichloromethane chloroform and mixtures thereof; and said isolated alcohol mixture has a purity level of 80-99% with respect to said nonalcoholic compounds contained in said beeswax.

3. A mixture of primary aliphatic alcohols isolated from beeswax; said mixture containing:

| | | |
|---|---|---|
| 1-eicosanol | C-20 | 0.0-5.0% |
| 1-docosanol | C-22 | 0.0-5.0% |
| 1-tetracosanol | C-24 | 15.0-30.0% |
| 1-hexacosanol | C-26 | 15.0-30.0% |
| 1-heptacosanol | C-27 | 0.0-5.0% |
| 1-octacosanol | C-28 | 12.0-22.0% |
| 1-tricontanol | C-30 | 20.0-35.0% |
| 1-dotriacontanol | C-32 | 5.0-15.0% |
| 1-tetratriacontanol | C-34 | 0.0-5.0% |

4. A pharmaceutical composition which comprises the mixture of claim 3 in combination with a pharmaceutically acceptable carrier, excipient or dilutant.

5. The composition of claim 4 in the form of a capsule, tablet, liquid or powder.

6. A method for treating or preventing hypercholesterolemia related diseases which comprises administering a pharmaceutically effective amount of the mixture of claim 4 to a human or mammal.

7. A method for reducing total cholesterol and LDL-C levels which comprises administering a pharmaceutically effective amount of the mixture according to claim 4 to a human or mammal in need thereof whereby said mixture inhibits cholesterol synthesis in the liver in the steps prior to mevalonate formation and increases the LDL catabolic rate which thereby increases hepatic LDL receptor activity.

8. A method of using the mixture of claim 3, which comprises administering said mixture as an antiplatelet, anti-inflammatory, anti-thrombotic or anti-ischemic agent to an individual in need thereof.

9. A method for preventing neurodegenerative disorders which comprises administering the mixture of claim 3 in a pharmaceutically acceptable amount to an individual in need thereof.

10. A method for improving male sexual activity which comprises administering the mixture of claim 3 to an individual in need thereof.

11. A method for at lowering LDL-C, total cholesterol, and improving LDL/HDL ratio which comprises administering the mixture of claim 3 in a pharmaceutically acceptable amount to an individual in need thereof.
